(19) United States
(12) Patent Application Publication
(10) Pub. No.: US 2003/0124548 A1
(43) Pub. Date: Jul. 3, 2003

(54) Method for association of genomic and proteomic pathways associated with physiological or pathophysiological processes

(76) Inventors: Christos Hatzis, Cambridge, MA (US); Pankaj Prakash, Malden, MA (US); John G. Babish, Brooktondale, NY (US); Linda M. Pacioretty, Brooktondale, NY (US)

Correspondence Address: KENYON & KENYON, One Broadway, New York, NY, 10004, US

(21) Appl. No.: 10/099,000

(22) Filed: Mar. 13, 2002

Related U.S. Application Data
(60) Provisional application No. 60/275,522, filed on Mar. 13, 2001.

(51) Int. Cl. C12Q 1/68; G01N 33/53; G06F 19/00; G01N 33/48; G01N 33/50

(52) U.S. Cl. 435/6; 435/7.1; 702/19

(57) ABSTRACT

The present invention provides methods for identifying relationships between gene expression and protein modifications in a cell by determining gene expression generated in the cell, determining protein modifications generated in the cell, and coordinating the gene expression and protein modifications generated in the cell. Also provided by the present invention is a computer system for identifying such a relationship between gene expression and protein modifications. The present inventive methods and computer systems are useful for investigating a variety of physiological or pathophysiological processes, including metabolic pathways, typing of diseased cells, and identifying biological activities of test materials.

### FIELD OF THE INVENTION

The present invention relates generally to functional genomics and proteomics, and more particularly, to methods for associating gene and protein data.

### BACKGROUND OF THE INVENTION

With the recent sequencing of the entire human genome and the accumulation of vast amounts of DNA sequences in databases, researchers are realizing that merely having complete sequences of genomes is not sufficient to elucidate biological function or pathology. Information buried in the human genome can be used to (1) identify genes that are central to cellular characteristics in each tissue; (2) define relationships among genes in specific cellular pathways; (3) examine genetic motifs on a physiological global scale; (4) type tumors using expression patterns to complement classical histology and predict disease development; (5) monitor the impact of a drug on a pathological state; and/or (6) assess potential toxicological effects of therapeutics.

A cell is normally dependent upon a multitude of metabolic and regulatory pathways for both homeostasis as well as survival. There is no strict linear relationship between gene expression and the protein complement or proteome of a cell.

In cells, the intricate relation between the synthesis of DNA, RNA and protein is circular and can be diagramed as presented in FIG. 1. DNA directs the synthesis of RNA, and RNA then directs the synthesis of protein; special proteins catalyze and regulate the synthesis and degradation of both RNA and DNA. This cyclic flow of information occurs in all cells and has been called the “central dogma” of molecular biology. Proteins are the active working components of the cellular machinery. Whereas DNA stores the information for protein synthesis and RNA carries out the instructions encoded in DNA, proteins carry out most biological activities; their synthesis and ultimate structure are at the heart of cellular function.

Messenger RNA (mRNA) encodes the genetic information copied from DNA in the form of a sequence of nucleotide bases that specifies a sequence of amino acids. The process of expressing the genetic information of DNA in the form of mRNA is termed transcription. On the other hand, translation refers to the whole procedure by which the base sequence of the mRNAs used to order and to join amino acids into a specific linear sequence of a protein; the resulting primary amino acid sequence is the initial determinant of protein structure.

Cellular identity and function are a direct result of both transcriptional and translational control processes. Since all cells possess identical genetic material, transcriptional control is necessary to differentiate one cell type from another. Transcriptional regulatory proteins, a family of DNA-binding proteins, control the expression of genes. Synthesis, processing and stabilization of mRNA by various enzymes and structural proteins represent additional controls to gene expression.

In addition to the variety of transcriptional controls developed by cells, ultimate protein functioning is dependent on several post-translational processes that affect protein structure and hence function. Proteolytic processing is employed to produce finished protein products from primary protein products. Other post-translational modifications include (1) farnsylation, phosphorylation and dephosphorylation; (2) protein-protein interactions to form homo or heteromeric complexes; and (3) intracellular compartment translocation.

Genomic and Proteomic Methods

The application of biotechnology to the understanding of gene structure and gene expression is defined as genomics. Currently one of the most active areas in molecular biology, genomics is providing enormous amounts of information regarding the composition of the human genome and transcriptional control. An underlying assumption in genomics is that gene expression as measured by mRNA is an accurate indicator of protein expression and functioning. However, studies on the relationship between mRNA abundance and protein expression have indicated that this association is less than 0.5.

Due to the poor association between transcription and the presence of mature, functional protein, a subset of genomics, termed proteomics, has developed that focuses specifically on the measurement of protein expression in the cell. Methods for measurement of cellular proteins are generally more laborious and have not been modified to provide high-throughput as have methods for the analysis of nucleic acids. Therefore, proteomic research lags far behind genomic research. While high throughput techniques have allowed for the development of data bases concerning transcriptional changes following exposure of cells to exogenous agents, the present state of knowledge as to how any exogenous agent perturbs protein expression and post-translational modification is such that not even experts in the field can estimate what changes will occur.

A cell is normally dependent upon a multitude of metabolic and regulatory pathways for homeostasis and adaptive responses. Since there is no strict linear relationship between gene expression and the protein complement of a cell, both gene and protein expression analyses are necessary to define critical cellular pathways in any biological process. Proteomics is complementary to genomics because it focuses on the gene products, which are the active agents in cells.

Proteomics is the large-scale study of proteins, usually by biochemical methods. The word proteomics has been associated traditionally with displaying a large number of proteins from a given cell line or organism on two-dimensional polyacrylamide gels. However, even when such gels can be run reproducibly between laboratories, determination of the identity of the protein is difficult. In the post-genomic era, protein identification may be affected through a number of laboratory techniques, including the following: (1) one-dimensional gels (with and without affinity purification), (2) two-dimensional gels, (3) micro-chips coated with antibodies, (4) non-denatured protein/protein complexes in solution; (5) post-translational modifiers such as phosphorylation or glycosylation; (6) functional assays for enzyme activity; (7) bioassays for cytokines or receptor/ligand binding; (8) localization of proteins within the cell; (9) large-scale mouse knockouts; (10) RNA interferences; (11) large-scale animal assays for functional proteins; and (12) differential display by two-dimensional gels.

Moreover, academic and commercial interest is moving from the genome to the proteome. There are three significant reasons for this movement. First, automated gene sequencing is reaching maturity as the emphasis expands from de novo sequencing. High-throughput automated DNA sequencing technologies have enabled sequencing of complex genomes. Second, understanding gene expression and protein interactions are likely to be more important than genomics. Researchers want to know what proteins are expressed and to what degree. As previously indicated, DNA expression is literally half the story. Altered protein and peptide expression is generally the key to understanding disease mechanisms. Finally, proteomics will engender a broader range of applications than genomics. In addition to the new areas in academic research and development proteomics will significantly affect drug discovery, preclinical research, clinical research, clinical diagnostics, veterinary medicine, forensics, agrochemical and naturaceuticals.

Information Management

Central to the integration of genomic and proteomic data is the application of sophisticated data handling and bioinformatic techniques to the large data sets characteristic of each methodology.

Efforts to characterize the gene expression patterns of the approximately 35,000 human genes are already producing large datasets. According to some estimates, in 3-5 years more than 10⁵ datasets will be available for analysis of the global gene expression patterns of the complete human genome. However, systems capable of analyzing and interpreting data collected from genomic-scale gene expression and proteomic studies are still in their infancy. Such systems will allow comparisons of the expression behavior of individual genes across tissues, developmental and pathological states, or responses to cellular perturbations. To enable these analyses, data warehousing systems are needed to support (1) data cleaning and verification; (2) integration of data from multiple sources; (3) consistent data models to standardize content of similarly named fields across databases, such as the Gene Expression Markup Language (GEML).

Statistical Methods for Analysis of Genomic Data

The advent of cDNA and oligonucleotide microarray technologies has led to a paradigm shift in biological investigation, such that the bottleneck in research is shifting from data gathering to data analysis. Considering the complexity of the genetic regulatory networks, predictive analysis of the expression patterns is not possible at genome-wide scale. Instead, exploratory analysis methods are typically employed to recognize any non-random patterns or structures in the data, which are then explained based on domain knowledge.

Several exploratory techniques have been recently used to interpret this mass of data. Among the most common, bottom-up hierarchical clustering algorithms use comprehensive pair-wise comparisons to determine similarly expressed genes. Results of these algorithms can be displayed in an intuitive way, but a number of limitations including poor scalability, a tendency to produce a large number of smaller clusters, and lack of global optimization due to the agglomerative nature of the algorithms limit their applicability in the analysis of large, complex datasets. Top-down clustering algorithms, such as k-means clustering, mixture components, and support vector machines, can produce globally optimal cluster structure and also allow the incorporation of prior knowledge to bias the clustering process. However, their application requires specification of number of cluster centers or prior examples to train the algorithms. Finally, projection clustering methods such as principal component analysis, multi-dimensional scaling and self-organizing maps have the advantages of eliminating redundant information and are computationally efficient, but the results could be difficult to interpret if the projection to lower dimensions is not biologically meaningful.

New classes of clustering techniques have been developed specifically for analyzing gene expression data. Of these, gene shaving is optimized for 2-way clustering and can be applied, for example, to find genes that vary the most across conditions. Another promising class of algorithms is the plaid clustering models, which allow for overlapping clusters and memberships in multiple clusters reflecting more realistically the multifunctional nature of many gene products.

Statistical Methods for Analysis of Proteomic Data

Perhaps due to the relatively limited availability of large-scale proteomics datasets, methods for analysis of proteomic patterns are not as well developed. At the exploratory level, the same methods used in gene expression analysis could be employed to detect patterns in proteomic profiles. The regulatory interactions between proteins can then be deduced from the clustering patterns of time-resolved measurements and captured using simple representations of genetic networks based on Boolean models.

Therefore, gene expression data are only a portion of the information necessary to accurately characterize cellular changes due to physiological adaptation, pathogenesis or exposure to xenobiotic agents. To fully understand the relationship between a cell and its environment, gene expression profiles must be determined; protein expression and associated post-translational modifications of proteins described; and changes in both gene expression and protein processing must be coordinated. Moreover, the association between gene expression and protein processing must be presented in a manner that allows for rapid identification of the relative involvement and interactions of numerous cellular pathways. At this time, no such process or methodology has been described in the literature.

An ideal process for the identification of genetic and proteomic pathways involved in homeostasis or pathophysiology would provide information and simultaneous analyses of both gene expression arrays and proteomic changes. Optimally, the procedure should condense the wealth of information generated into summary statistics that are biologically relevant and easy to understand. Furthermore, the process should be applicable to a variety of techniques for the measurement of gene expression arrays as well as protein processing.

### SUMMARY OF THE INVENTION

The present invention provides methods of identifying relationships between gene expression and protein modifications in a cell by determining gene expression generated in the cell, determining protein modifications generated in the cell, and coordinating the gene expression and protein modifications generated in the cell. Also provided by the present invention is a computer system for identifying the relationship between gene expression and protein modifications having (1) a database including records of gene expression data and protein modifications data, (2) one or more algorithms for statistically analyzing the gene expression and protein modifications data, (3) one or more algorithms for coordinating the statistically analyzed gene expression and protein modifications data, (4) a system for output and presentation of the results from the algorithms, (5) a repository systems to index and stored the database and results, and (6) a query system for retrieval of database and results.

### BRIEF DESCRIPTION OF THE DRAWINGS

FIG. 1 provides a schematic diagram illustrating the relationship of gene expression to the production of the functionally active protein product.

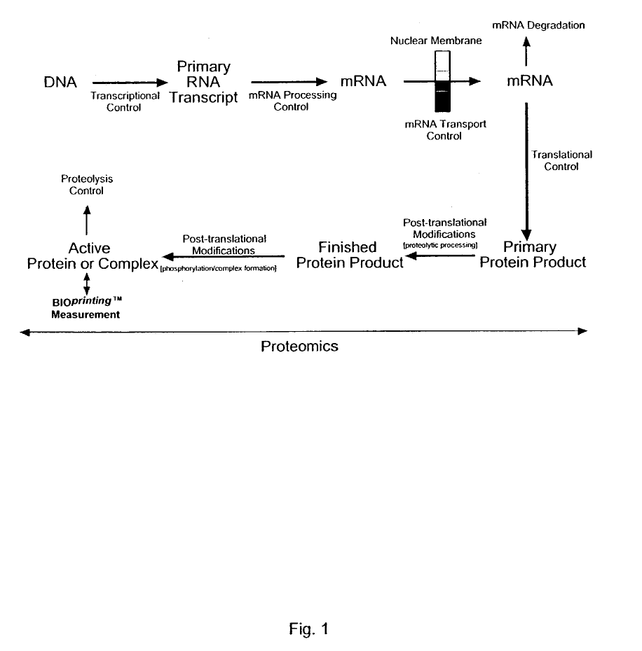

FIG. 2 schematically illustrates a typical system for the identification of gene expression using synthetic oligonucleotides attached to a microchip containing 65,000 to 250,000 oligos, each represented in 10⁷ to 10⁸ full-length copies.

FIG. 3 graphically illustrates cluster homogeneity plots for wild-type (WT) and mutant (F5) gene expression profiles. Both curves are very similar indicating an almost identical structure in the global expression patterns of the two strains.

FIG. 4 graphically illustrates Euclidian length of vectors of expression levels relative to control for genes in each cluster vs cluster size. The filled circle represents the entire set of genes.

FIG. 5 graphically illustrates expression signatures of individual clusters. Each chart shows the average expression profile of the genes in the given cluster. Error bars are equal to one standard deviation. The average expression profile of the entire set of genes is also shown for comparison. FIGS. 5A-D graphically represent early up-regulated gene clusters as compared to the population: cluster 12 (FIG. 5A), cluster 20 (FIG. 5B), cluster 35 (FIG. 5C), and cluster 19 (FIG. 5D). FIGS. 5E-I graphically represent late up-regulated gene clusters: cluster 18 (FIG. 5E), cluster 16 (FIG. 5F), cluster 14 (FIG. 5G), cluster 15 (FIG. 5H), and cluster 17 (FIG. 5I). FIGS. 5J-N graphically represent down-regulated gene clusters: cluster 6 (FIG. 5J), cluster 4 (FIG. 5K), cluster 1 (FIG. 5L), cluster 10 (FIG. 5M), and cluster 22 (FIG. 5N).

FIG. 6 charts the classification of gene clusters according to common expression signatures.

FIG. 7 schematically illustrates a comparison of the immediate early genes (IEGs) (FIG. 1A) and late up-regulated genes (FIG. 1B) for wild type and F5 mutant strain (see Table 2 for annotations).

FIG. 8 illustrates an array of the Pearson correlation coefficients for the expression profiles of corresponding genes from the wild type and mutant strains. Brighter red indicates higher positive correlation, green negative, and black indicates near-zero correlation.

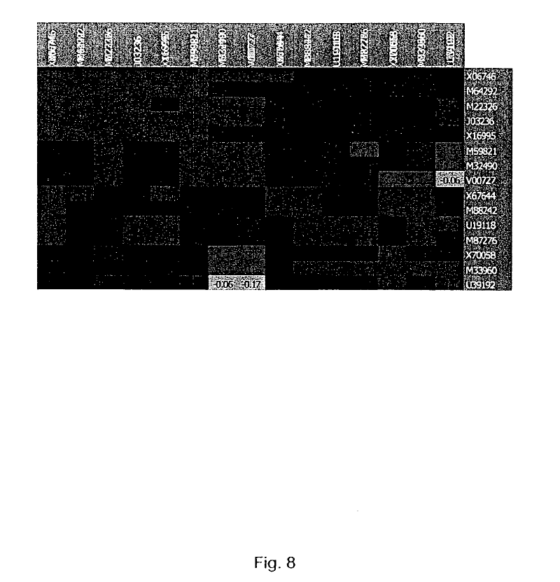

FIG. 9 schematically illustrates the present inventive methods of determining a genomic expression profile and a proteomic expression profile and correlating the results of each profile.

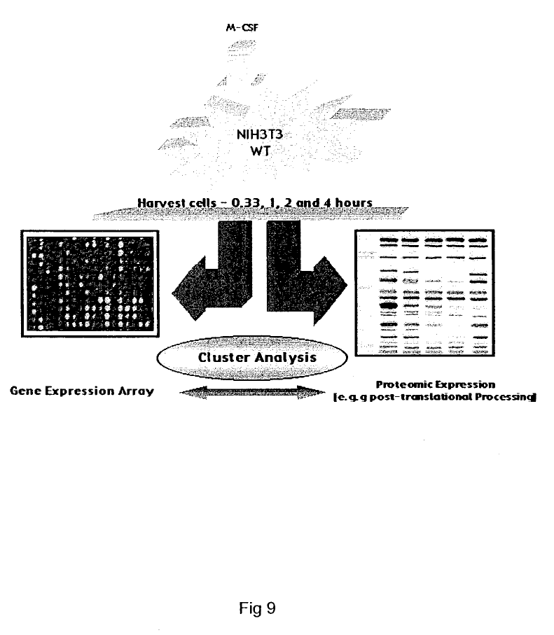

FIG. 10 is a gel showing time-associated changes in phosphotyrosyl protein expression in test cells following incubation with test material.

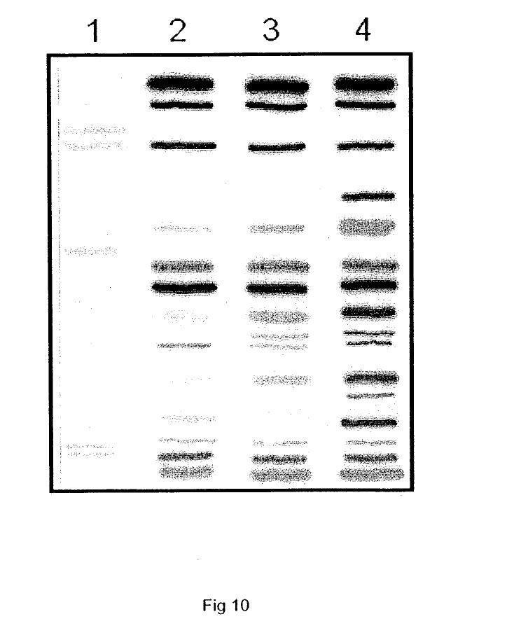

FIG. 11 charts distribution of proteomic clusters in test cells following incubation with test material.

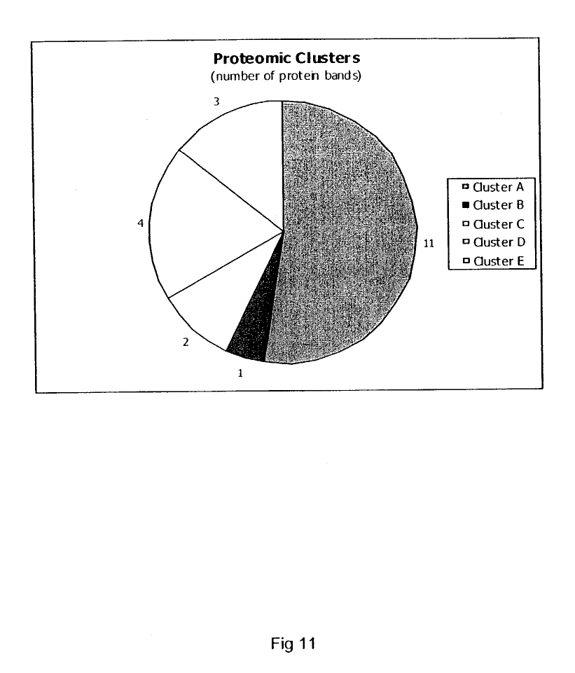

FIG. 12 graphically represents signature profiles of proteomic clusters in test cells following incubation with test material as compared to the population: cluster E (FIG. 12A), cluster C (FIG. 12B), cluster B (FIG. 12C), cluster D (FIG. 12D), and cluster A (FIG. 12E).

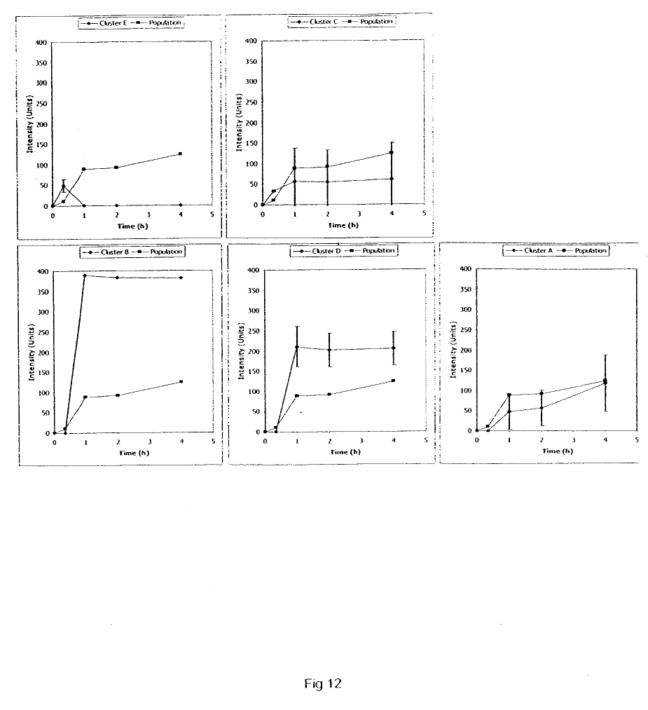

FIG. 13 provides associations of gene expression and proteomic clusters based on the Pearson's correlation coefficient between the profiles in test cells following incubation with test material.

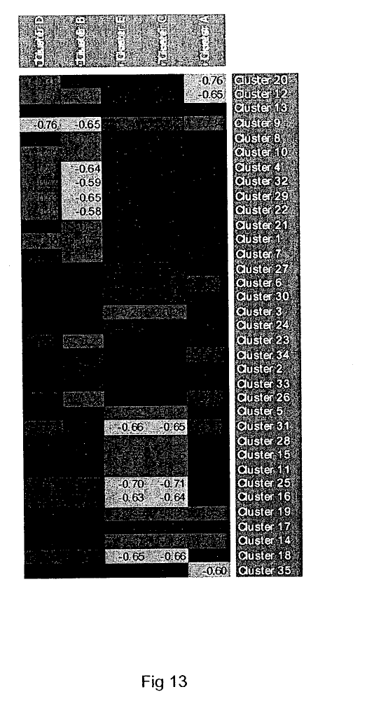

FIG. 14 schematically illustrates the signaling pathway with the highest degree of association in test cells following incubation with test material, which is the G1 progression phase of the cell cycle, and the cell cycle regulatory proteins identified by the present invention.

### DETAILED DESCRIPTION OF THE INVENTION

Ascertaining the impact of uncharacterized perturbations on the cell is a fundamental problem in biology. The invention relates to a method for the measurement and modeling of numerous complex cellular functions and interactions. It is not necessary to construct reference gene or protein expression databases or refer to previously developed libraries of expression.

Accordingly, the present invention provides a method of identification of functionally relevant metabolic networks, proteomic alterations or signaling pathways. The invention incorporates functional aspects of proteins and protein processing such as, for example, phosphorylation, farnsylation, methylation, and any post-translational processing as well as subcellular localization and intracellular trafficking, and overcomes shortcomings of prior modeling systems.

The present invention is further directed to a method of identification of gene expression comprising the use of a gene array, composed of several hundred to tens of thousands of genes, capable of discerning the expression of genes within a biological cell.

Furthermore, the method is applicable to a variety of techniques for the measurement of gene expression and quantification of proteins and protein processing. For example, using common measurement techniques such as one- or two-dimensional gel electrophoresis, databases of, for example, 2,000 or more proteins can be generated and analyzed.

The present invention further provides a statistical procedure for the individual analysis of gene and protein expression or modification.

The methods provide a useful interface for biological and statistical techniques and allow for the identification and quantification of gene expression and protein information separately or simultaneously.

In an embodiment of the invention, algorithms are provided for determining linkages and associations between gene expression and protein processing. In a preferred embodiment, experimental data from gene expression arrays and protein processing are presented as connected biological signaling or metabolic pathways. Within each relationship, probability statements can be included that allow the researcher to weigh the relative contribution of each signaling pathway among a group of arbitrarily chosen signaling pathways representative of all biological aspects of cellular functioning.

The present invention is also directed to a method for describing the metabolic or signaling changes induced by a test material or physiological process which includes, subjecting a eukaryotic cell to the test material, lysing the tested eukaryotic cell, isolating the DNA or mRNA and protein of the cell, performing a mathematical cluster analysis with the gene and protein expression data that includes developing relationships between gene expression and functional changes in cellular proteins. The test material can be a single endo- or exomolecule or any combination of mixtures of endo- and exomolecules. The physiological process may be cell synchronization, starvation, aging or contact inhibition.

The invention provides an analytical system, preferably computer-based, to estimate and describe the most probable network of biological responses to endogenous agents, such as hormones, cytokines and neurotransmitters, that modify proteins in any metabolic pathway. In addition, the present invention provides an analytical system, again preferable computer-base, to estimate and describe the most probable network of biological responses or biological activity of xenobiotic compounds (test compounds), such as drugs, food ingredients, environmental pollutants and toxins. This complete computational process would consist of a system for the generation of gene and protein expression data from eukaryotic cells and statistical techniques that can identify gene and protein clusters that suggest probable pathways and networks, of molecular signaling. Thus, the present invention provides analytical methods that may be used, for example, in drug design, application of genome and proteome information, and analysis of chemical safety.

Gene expression profiles and/or protein modification or expression profiles are developed using iterative global partitioning clustering algorithms and Bayesian evidence classification to identify and characterize clusters of genes, proteins and genes and proteins having similar expression profiles. Protein expression is characterized using one- or two-dimensional separation techniques and post-translational processing is assessed with antibody or chemical detection of protein modifications such as phosphorylation, acetylation, farnsylation or methylation. Cell processing techniques such as differential centrifugation may also be used.

Finally, the present invention employs a knowledge based, statistical technique that can suggest probable pathways and networks of molecular signaling from the above-identified gene and protein clusters.

It is to be understood that this invention is not limited to the particular configurations that are exemplified, as process steps and materials may vary. It is also to be understood that the terminology employed herein is used for the purpose of describing particular embodiments only and is not intended to be limiting since the scope of the present invention will be limited only by the appended claims and equivalents thereof.

It must be noted that, as used in this specification and the appended claims, the singular forms “a,” “an,” and “the” include plural referents unless the context clearly dictates otherwise.

The present invention relates to methods of identifying a relationship between gene expression and proteomic modifications in a cell by determining gene expression generated in the cell, determining proteomic modifications generated in the cell, and coordinating the gene expression and proteomic modifications generated in the cell.

According to the present invention, the type and amount of structural changes that affect cellular proteins when the cell undergoes physiological changes or is exposed to a compound or mixture of compounds is related to coordinate changes between genes and proteins and among proteins along predetermined signaling pathways. Moreover, according to the present invention, the type and amount of structural changes observed in eukaryotic cellular proteins and the signaling pathways in which they function are reproducible. This means that physiological changes or the amount of biological or pharmacological activity of a compound, or an extract or mixture of compounds, can be determined by quantifying structural changes induced in cellular proteins and determining the effect on signaling pathways in which they operate in vivo or in cultured cells. Similarly, the activity of one preparation of a compound, or an extract or mixture containing several compounds, can be compared against that of another preparation, for example, a control preparation.

The present inventive methods also provide for identification of biological activity of one or more test materials. Such identification can be accomplished by exposing a cell to the one or more test materials and identifying the relationship between gene expression and protein modifications generated in the cell in response to exposure to the one or more test materials according to the present inventive methods.

One of the benefits of the present invention is that this assessment of biological or pharmacological activity can be performed on one or more compounds, a mixture of compounds, under a variety of physiological conditions without the need to identify which component or components provide the activity. A combination of compounds can also be compared using the present inventive methods. The present invention is therefore particularly useful for assessing the activity of complex mixtures which may contain one or more components, that separately have little or no activity, but that have significant activity in combination with other components.

Thus, an embodiment of the present invention is directed to a method for establishing the biological activity of a test material or describing physiological changes by assessing whether structural changes that are induced in proteins present in the eukaryotic cell are coordinate with alterations in gene expression. The functional properties of a cell can be assessed by analyzing the state of the cellular proteins.

In an additional embodiment, the present inventive methods can be utilized to investigate a metabolic pathway. To accomplish this, a cell is exposed to an agent involved in the metabolic pathway and the relationship between gene expression and protein modifications generated in the cell in response to the agent is identifying according to the method of present invention. Any suitable agent that alters the metabolic pathway can be used in the context of the present invention. It should be appreciated that the agent used in this embodiment can be native or foreign to the cell to which it is exposed. Moreover, any metabolic pathway can be investigated using these methods. Such investigation is detailed in the examples. In examples, macrophage-colony stimulating factor (M-CSF) is the agent to which wild-type and mutant strains of NIH 3T3 mouse fibroblast cells are exposed. M-CSF stimulates receptor tyrosine kinase pathways, the metabolic pathways being investigated.

In one other embodiment, the present invention provides a method for determining whether a test material affects cellular signaling pathways by incubating the test material with cultured mammalian cells to produce treated test cells, lysing the treated test cells, characterizing gene expression and protein tyrosine phosphorylation, and establishing clusters of genes and phosphotyrosyl proteins and coordinate clusters of genes with phosphotryosyl proteins. Results of the cluster analysis are used to generate a model of coupling pathways between or around specific biomolecules as a result of exposure to the test material. These pathway results may be compared to control cells that have not been exposed to the test material. Alternatively, the control cells may be cultured mammalian cells that are in a quiescent state or non-dividing condition. Control cells can also be treated cells that exhibit no physiological response or a physiological response different from that of the treated test cell.

The methods of the present invention can also be used to determine the type of diseased cell. In this embodiment, the relationship between gene expression and protein modifications in the diseased cell is identifying according to the present inventive methods. Then, the relationship between gene expression and protein modifications in a corresponding normal cell is also identified according to the present inventive methods. Finally, the coordinated gene expression and protein modifications of the diseased cell are compared with that of the normal cell. In this manner, any type of diseased cell can be assayed. For example, a cancer cell can be assayed for various clinical markers, as well as various potential therapeutic targets. When a patient's diseased cell is assays, such a determination may allow clinicians to better treat the individual.

According to the present invention, any primary or immortalized cell line may be used. As used herein, examples of primary cell lines include cancerous and non-cancerous cells derived from any tissue specimen, for example, from mesangial, embroyonic, brain, lung, breast, uterine, cervical, ovarian, prostate, adrenal cortex, skin, blood, bladder, gastrointestinal, colon and related tissues. Example of immortalized mammalian cell lines that can be used in the present methods include human LNCaP prostate, human HeLa, colon 201, neuroblastoma, retinoblastoma and KB cell lines, and mouse 3T3, L and MPC cell lines. Immortalized cell lines may be obtained from recognized cell repositories, for example, the American Type Culture Collection. Cells may also be obtained from treated or exposed humans, mice, dogs or nonhuman primates, or other animals.

Determination of gene expression, or gene expression analysis, may be accomplished by any one of many suitable procedures available in the art. Examples of such methods may employ microchip arrays of genes, northern blot analysis of gene transcription, or analysis of chemically modified nucleic acids. In addition, determination of gene expression may be accomplished by Serial Analysis of Gene Expression (SAGE). See generally Yamamoto et al., J. Immunol. Methods, 250(1-2): 45-66 (April 2001).

For example, mRNA (˜1 μg) is isolated from the test eukaryotic cells to generte first-strand cDNA by using a T7-linked oligo(dT)primer. After second-strand synthesis, in vitro transcription (Ambion) is performed with biotinylated UTP and CTP (Enzo Diagnostics), the result is a 40- to 80-fold linear amplification of RNA. Forty micrograms of biotinylated RNA is fragmented to 50- to 150-nt size before overnight hybridization to Affymetrix (Santa Clara, Calif.) HU6000 arrays. Arrays contain probe sets for 6,416 human genes (5,223 known genes and 1,193 expressed sequence tags (EST)). Because probe sets for some genes are present more than once on the array, the total number on the array is 7,227. After washing, arrays are stained with streptavidin-phycoerythrin (Molecular Probes) and scanned on a Hewlett Packard scanner. Intensity values are scaled such that overall intensity for each chip of the same type is equivalent. Intensity for each feature of the array is captured using the GENECHIP SOFTWARE (Affymetrix, Santa Clara, Calif.), and a single raw expression level for each gene is derived from the 20 probe pairs representing each gene by using a trimmed mean algorithm. A threshold of 20 units is assigned to any gene with a calculated expression level below 20, because discrimination of expression below this level is not performed with confidence in this procedure.

After establishing the gene expression for the test cells, gene expression profiles are analyzed using suitable statistical analyses, for example, iterative global partitioning clustering algorithms and bayesian evidence classification, to identify and characterize clusters of genes having similar expression profiles. See, e.g., Long et al., J. Biol. Chem., 276(23): 19937-44 (June 2001). Any suitable clustering algorith can be utilized in the context of the present invention, including the various clustering algorithms and methods described previously.

The steps involved in this statistical analysis are (1) determination of the fold induction (log ratio) of the genes, (2) normalization of the gene profile to a magnitude equal to 1, (3) partition clustering of all genes measured in to determine unique clustering patterns, (4) differentiation of gene clusters in each test populations into the following sub-groups based on their expression as compared to the population-average profile: early up-regulated, late up-regulated, down-regulated and others, (5) performance of a comparative analysis to explore the common genes in the early up-regulated and down-regulated cluster sub-groups in the test populations of cells, and (6) correlation based on the Pearson correlation coefficient to determine differences and similarities among the sub-groups in the test populations of cells.

According to the present inventive methods, protein modifications (proteomics) involves the qualitative and quantitative measurement of gene activity by detecting and quantitating expression at the protein level, rather than at the messenger RNA level. Protein modifications can also involve non-genome-encoded events including the post-translational modification of proteins (including phosphorylation, glycosylation, methylation, and/or farnsylation), interactions between proteins, and the location of proteins within the cell. The structure, function, or levels of activity of the proteins expressed by a cell are also of interest. Essentially, protein modifications involve part or all of the status of the total protein contained within or secreted by a cell.

According to the present invention, any method used to study post-translational changes in cellular proteins can be used to assess whether these changes have occurred in cellular proteins. Such changes also include, for example, protein amounts, protein-protein interactions and covalent modifications. The nature and extent of functional or structural changes induced in the cellular proteins of mammalian cells tested by exposure to a test material may be determined by any procedure available to one of skill in the art.

Any suitable method thus can be used to determine protein modifications. Protein modifications can also be determined using one-dimensional gel electrophoresis, with or without affinity purification, differential display by two-dimensional gels, microchips coated with antibodies, functional assays for enzyme activity and bioassays for cytokines or receptor/ligand binding. The protein modifications can also determined by identification of non-denatured protein/protein complexes in solution, localization of the proteins within the cell, and through large-scale animal assays for functional proteins.

These suitable methods further include isotope-coded affinity tags, protein chips, microfluidics, and differential in gel electrophoresis, for example. Using isotope-coded affinity tags, specific proteins in two separate samples can be chemically tagged with distinct heavy and light isotopes. By tracking the relative abundance with a mass spectrometer, a quantitative measure of protein expression changes can be determined. On protein chips, a checkerboard-like grid of molecules designed to capture specific proteins at specific sites is laid down. Fluorescent probes or other means of detection are used to determine where proteins bind on the grid. Because the identity of the probes at each spot on the grid is known, this reveals which protein is captured. In microfluidics, silicon, glass, and plastic chips, engineered to harbor networks of sample holders, channels, and reaction chambers to carry out the complex sequence of steps needed to prepare protein samples for analysis by mass spectrometers and other analytical equipment, have been developed. Because the chips are fast and can work with very small samples, they have the potential to dramatically improve the speed and sensitivity of proteomic analyses. Finally, with differential in gel electrophoresis, a global view of how protein expression changes between two samples is determined. Proteins from one sample are all tagged with a single fluorescent compound, while those from another sample are tagged with a different colored fluorescent dye. The two samples are then mixed together and the individual proteins are separated on a single two-dimensional gel; this separates proteins in one direction by their charges and in the perpendicular direction by their molecular weights. A quick look at the gel reveals whether separate spots show both colors—or just a single color that shows which sample harbors the protein.

In one particular example, to identify the types of proteins that may have undergone functional changes, a cell lysate of a tested population of eukaryotic cells can be separated under either denaturing or non-denaturing conditions. Non-denaturing conditions are used for observing protein-protein interactions. Denaturing conditions facilitate reproducible identification of individual protein species and are preferred when identifying changes in the type and amount of protein phosphorylation. Separation of both protein-protein complexes and individual proteins may be accomplished by any available chromatographic or electrophoretic procedure. For example, cellular proteins can be separated by size and/or charge using gel exclusion, ion chromatographic, reverse phase, electrophoretic (one or two dimensional) or other procedures. See, e.g., Sambrook et al. Molecular Cloning: A Laboratory Manual, Vols. 1-3 (Cold Spring Harbor Press, N.Y., 1989).

After separation, the cellular proteins that may have undergone functional (structural) changes can be visualized by any procedure available in the art. Procedures and reagents for visualizing proteins are well known in the art and include, for example, staining with dyes that bind to proteins and reacting the proteins with antibodies that have a covalently attached reporter molecule. Phosphorylated proteins can be visualized by reacting the cellular proteins with monoclonal antibodies directed against the phosphorylated serine, threonine or tyrosine amino acids that are present in the proteins. For example, monoclonal antibodies useful for isolating and identifying phosphotyrosine-containing proteins are described in U.S. Pat. No. 4,53,439 to Frackelton et al. (issued Sep. 24, 1985), and Schieven et al., Lineage-specific Induction of B Cell Apoptosis and Altered Signal Transduction by Phosphotyrosine Phosphatase Inhibitor Bis(maltolato)oxovanadium (IV), 270, J.Biol.Chem. 20824(1995). The procedures and reagents provided in these references can readily be adapted by one of skill in the art to practice the methods of the present invention.

Antibodies used for visualizing cellular proteins can be labeled by any procedure known in the art, for example by incorporation of a “reporter molecule” by covalent bonding or other means to the antibody or antibody detection agent.

A reporter molecule, as used herein, is a molecule that provides an analytically identifiable signal allowing one of skill in the art to identify when an antibody has bound to the protein to which it is directed. Detection may be either qualitative or quantitative. Commonly used reporter molecules include flurophores, enzymes, biotin, chemiluminescent molecules, bioluminescent molecules, digoxigenin, avidin, streptavidin or radioisotopes. Commonly used enzymes include horseradish perodicsas, alkaline phosphatase, glucose oxidase and beta-galactosidease among others. The substrates to be used with these enzymes are generally chosen for the production, upon hydrolysis by the corresponding enzyme, of a detectable color change. For example, p-nitrophenyl phosphate is suitable for use with alkaline phosphatase reporter molecules for horseradish peroxidase, 1,2-phenylenediamine, 5-aminosalicylic acid or toluidine. Incorporation of a reporter molecule onto an antibody can be by any method known to the skilled artisan.

After separation and visualizing the proteins, the amount of each protein species may be assessed by readily available procedures. For example, the proteins may be electrophoretically separated on a polyacrylamide gel, and after staining the separated proteins, the relative amounts can be quantified by assessing its optical density.

Data analysis for functional protein expression is then conducted in a manner similar to that for gene expression analysis. For each protein band intensity measurements are first normalized to magnitude of 1 across the time profile. Data can also be normalized across protein bands to a magnitude of 1 at each time point. Partitioning k-means clustering is applied to the normalized data. Average profiles are calculated for the proteins within each cluster. Protein clusters are grouped according to the dynamics accumulation to early or late phosphorylated clusters. The similarity of the proteomic clusters to the genomic expression clusters is then determined through association analysis based on a similarity measure, as for example the Pearson's correlation coefficient or Euclidean distance of the two profiles. Coordination of such data, as understood by a skilled artisan, would encompass any and all types of suitable comparisons or analyses to determine the differences, similarities, and/or relationships between gene expression and protein modification, resulting in a more complete understanding of the activities occurring within a cell.

Current popular methods described in the prior art for determining the identity of proteins undergoing expression alterations in cells, such as mass spectral analysis, suffer from the inability to identifiy post-translational modifications reproducibily or at all. Thes methods, therefore, lack sufficient robustness for the identification of putative cell signaling networks related to test variables within an experiment. This limitation has been underscored more dramatically with the completion of the mapping of the human genome. For example, while a one gene—one protein relationship is generally true for lower organisms, in more complex forms of live such as humans, a process of alternative splicing is employed to create an extremely complex array of proteins from the 25,000 to 35,000 genes that are now estimated to exist in the human genome. Furthermore, it is also estimated that only 5% of the human genome contains genes used to build proteins. Correlating these recent findings with the estimate that there exists 150,000 to 350,000 functional proteins in the human organism, it becomes clear that functional assessment of proteins is essential for the elaboration of metabolic or signaling networks responding to the test conditions.

It is a further object of the present invention to provide a dataset of known proteins that includes, for example, their molecular weights, known post-translational processing, such as phosphorylation, methylation, acetylation or complex formation, and functional categories, such as apoptosis, cell cycle regulation, proliferation, secretion and transcription factor. Known interactions with other proteins for each functional category are also noted. Minimally this dataset should contain 2,000 proteins.

According to the present invention, networks of signaling pathways may be inferred by searching the protein database with the results of gene/protein clustering analysis previously described. As a result of the methods of the present invention, pathways that connect molecules of interest can be retrieved from the functional protein dataset based upon biological attributes, functions, sequences and the structure of the molecules identified in the gene/protein cluster analysis. The retrieved pathways are represented as graphs consisting of nodes and arrows. Each node represents a functional match from the input cluster analysis. Probability statements relating the degree of concordance with the cluster analysis over the length of the graph may be used to describe the likelihood of the involvement of a particular pathway with the test variables. This method of representation differs from the prior art in the use of functional nodes (data hits) in graphic representations.

The present invention further provides a computer system for identifying the relationship between gene expression and protein modifications. Such a computer system includes (1) a database having records of gene expression data and protein modifications data, (2) one or more algorithms for statistically analyzing the gene expression and protein modifications data, (3) one or more algorithms for coordinating the statistically analyzed gene expression and protein modifications data, (4) a system for output and presentation of the results, (4) a repository systems to index and stored the database and results, and (5) a query system for retrieval of database and results.

Another computer-based system for predicting the relationship between gene expression and functional protein expression is provided by the present invention. This system involves the following: (1) a database management system for storing gene expression data and protein modification data; (2) a database system for aggregating information about individual genes and proteins, including chromosomal location, function, pathway membership, phosphorylation status; (3) algorithms for correcting experimental data for experimental biases; (4) one or more clustering algorithms for extracting patterns from gene expression profiles, and from functional protein expression profiles; (5) one or more algorithms for extracting relationships between gene expression patterns and functional protein expression patterns; (6) algorithms for annotating gene expression profiles to derive functional characterization of gene expression or protein expression response; (7) a repository for storage of derived relationships; and (8) a query system for retrieval of discrete patterns, relationships and experimental conditions.

The following examples are intended to illustrate but not in any way limit the invention.

### EXAMPLES

### Example 1

The present example demonstrates gene induction by ligand-stimulated receptor tyrosine kinases (RTKS) in fibroblast cells

Receptor Tyrosine Kinases (RTKs) transduce extra-cellular signals that trigger important cellular events, such as mitosis, development, wound repair, and oncogenesis. When bound by ligand(s), RTKs mediate these responses by activating a variety of intracellular signaling pathways. Such signaling pathways result in the transcription of a set of “Immediate Early Genes” (IEGs). IEG products initiate cellular processes that depend on protein synthesis, such as mitogenesis. Wild-type and mutant strains of NIH3T3 mouse fibroblast cells are stimulated with macrophage-colony stimulating factor (M-CSF) for various time points, and the M-CSF-activated signaling pathway-induced gene expression is determined. The essential objective of the study is to characterize the RTK-mediated interactions between the intracellular signaling pathways.

Experimental Methodology

The following equipment used for experiments in this Example includes an Ohaus Explorer analytical balance, (Ohaus Model #EO1140, Switzerland), biosafety cabinet (Forma Model #F1214, Marietta, Ohio), pipettor, 100 to 1000 μL (VWR Catalog #4000-208, Rochester, N.Y.), cell hand tally counter (VWR Catalog #23609-102, Rochester, N.Y.), CO₂ Incubator (Forma Model #F3210, Marietta, Ohio), hemacytometer (Hausser Model #1492, Horsham, Pa.), inverted microscope (Leica Model #DM IL, Wetzlar, Germany), pipet aid (VWR Catalog #53498-103, Rochester, N.Y.), pipettor, 0.5 to 10 μL (VWR Catalog #4000-200, Rochester, N.Y.), pipettor, 100 to 1000 μL (VWR Catalog #4000-208, Rochester, N.Y.), pipettor, 2 to 20 μL (VWR Catalog #4000-202, Rochester, N.Y.), pipettor, 20 to 200 μL (VWR Catalog #4000-204, Rochester, N.Y.), PURELAB Plus Water Polishing System (U.S. Filter, Lowell, Mass.), Refrigerator, 4° C. (Forma Model #F3775, Marietta, Ohio), vortex mixer (VWR Catalog #33994-306, Rochester, N.Y.), a water bath (Shel Lab Model #1203, Cornelius, Oreg.), microfuge tubes, 1.7 mL (VWR Catalog #20172-698, Rochester, N.Y.), pipet tips for 0.5 to 10 μL pipettor (VWR Catalog #53509-138, Rochester, N.Y.), pipet tips for 100-1000 μL pipettor (VWR Catalog #53512-294, Rochester, N.Y.), pipet tips for 2-20 μL and 20-200 μL pipettors (VWR Catalog #53512-260, Rochester, N.Y., pipets, 10 mL (Becton Dickinson Catalog #7551, Marietta, Ohio), pipets, 2 mL (Becton Dickinson Catalog #7507, Marietta, Ohio, pipets, 5 mL (Becton Dickinson Catalog #7543, Marietta, Ohio) and a cell scraper (Corning Catalog #3008, Corning, N.Y.)

Chemicals, reagents and buffers necessary include dimethylsulfoxide (DMSO) (VWR Catalog #5507, Rochester, N.Y.), Modification of Eagle's Medium (DMEM) (Mediatech Catalog #10-013-CV, Herndon, Va.), fetal bovine serum, Heat Inactivated (FBS-HI) (Mediatech Catalog #35-011-CV, Herndon, Va.), Penicillin/Streptomycin (Mediatech Catalog #30-001-CI, Herndon, Va.), murine fibroblast cells (American Type Culture Collection Catalog #TIB-71, Manassas, Va.), tissue culture plate, 24-well, 3.4 mL capacity (Becton Dickinson Catalog #3226, Franklin Lanes, N.J.) and ultra-pure water (Resistance=18 megaOhm xcm deionized water).

Murine 3T3 cells (ATCC Number CCL-92) are grown in DMEM with 10% FBS-HI with added penicillin/streptomycin and maintained in log phase prior to experimental setup. To make growth medium, to a 500 mL bottle of DMEM, add 50 mL of heat inactivated FBS and 5 mL of penicillin/streptomycin. Store at 4° C. Warm to 37° C. in water bath before use.

Cell Surface Receptor Modification

A chimeric growth factor receptor having the signaling activity of M-CSFR and activated by binding macrophage colony stimulation factor (M-CSF), referred to as “wild-type” chimeric receptor (ChiR(WT)) is constructed using standard procedures in molecular biology. Also, a mutant strain ChiR(F5)-3T3 is constructed employing accepted site-directed mutagenesis techniques.

Gene induction in the wild-type strain is determined. ChiR(WT)-3T3 cells are stimulated with M-CSF alone and in combination with cycloheximide (CHX) to assess which induced genes behave as IEGs and which require protein synthesis for their induction. M-CSF treatments were 40 ng/ml in 0.5% bovine calf serum media for 20 min, 1 hr, 2 hr and 4 hr. CHX treatments were 10 μg/ml for 4 hr. Gene induction in the mutant strain is also determined. The F5 mutant strain is stimulated with M-CSF for 20 min, 1 hr, 2 hr and 4 hr.

Gene expression levels are measured using oligonucleotide arrays (Affymetrix) containing detectors for 5938 mouse genes and EST sequences. To be classified as an IEG in the wild-type strain, genes had to be induced by M-CSF in the presence and absence of CHX. Sixty-six genes met the criteria for being IEGs and an additional 43 genes are induced by M-CSF+CHX but are not strongly induced by M-CSF alone.

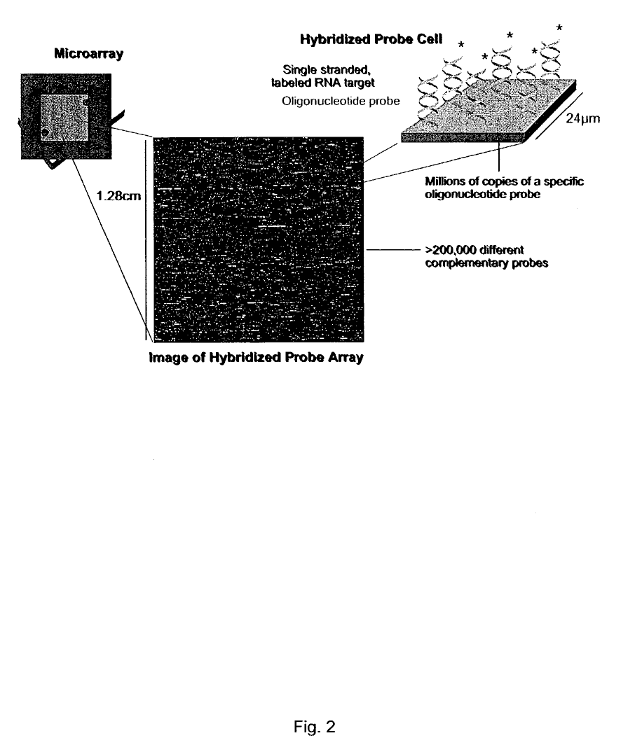

The RNA is used for expression monitoring, using oligonucleotide arrays (Affymetrix, Inc.) containing detectors for 5938 genes and EST sequences (FIG. 2). It should be noted that although changes in transcript abundance are not necessarily due to transcriptional upregulation, previous experiments have shown that transcriptional upregulation is by far the preponderant model if IEG induction by RTKs.

To initially identify a set of clear IEGs, stringent criteria are set including at least a 2-fold induction in both replicate studies and at least 3-fold induction in one of the replicates at one time point. Although the oligonucleotide arrays monitor less than 10% of the total number of mouse genes, the 66 IEGs probably represent a much larger proportion of the total number, because of extensive discovery efforts for this class of genes.

Protein quantification was determined from cell lysates using a Packard FluoroCount Model #BF10000 fluorometer (Meriden, Conn.). Other equipment not previously listed included a Forma Model #F3797 −30° C. freezer, Heating Block (VWR Catalog #13259-030, Rochester, N.Y.), Microfuge (Forma Model #F3590, Marietta, Ohio). The procedure described in the NanoOrange Protein Quantitation Kit (Molecular Probes Catalog #N-6666, Eugene, Oreg.) is followed without modification.

Gene expression profiles were analyzed using iterative global partitioning clustering algorithms and bayesian evidence classification to identify and characterize clusters of genes having similar expression profiles. Since the dynamics of the expression profiles are important in elucidating the functional role of the genes, the entire time series of expression measurements for each gene was used in the analysis.

The steps involved are as follows:

1) Determine the fold induction (log ratio) of the genes in the wild-type and mutant strains for each time point relative to the control at 0 hr (no stimulation)

2) Normalize gene profiles to magnitude equal to 1

3) Conduct partitioning clustering on 6312 genes in each strain to determine unique clustering patterns

4) Differentiate gene clusters in each strain into the following sub-groups based on their expression as compared to the population-average profile: early up-regulated, late up-regulated, down-regulated and others

5) Carry-out a comparative analysis to explore the common genes in the early up-regulated and down-regulated cluster sub-groups in the two strains

6) Conduct correlation analysis based on the Pearson correlation coefficient to determine differences and similarities among the IEGs in the two strains.

Intermediate Early Genes Induced by M-CSF Treatment of NIH3T3 Cells

The IEGs induced by 40 ng/mL M-CSF stimulation of quiescent NIH3T3 WT and F5 mutant cells are listed in Table 2.1 by time of peak observed induction. Each gene is classified as previously reported if it has been reported to be M-CSF or serum inducible in fibroblasts.

TABLE 2.1
Intermediate Early Genes Induced By M-CSF Treatment of NIH3T3 Cells

| | Functional | | Fold Induction |
|---|---|---|---|
| Encoded Protein | Classification | Peak Time Point | WT/F5 |
| Zif/268 | Transcriptional | 20 min | 72.1/43.5 |
| c-Fos | Transcriptional | 20 min | 54.0/26.5 |
| Pip92 | Cytoplasmic reg | 20 min | 36.3/12.6 |
| Cyr61 | Secreted | 20 min | 29.5/10.7 |
| JunB | Transcriptional | 20 min | 23.9/8.4 |
| Krox20 | Transcriptional | 20 min | 12.4/5.6 |
| MKP1 | Cytoplasmic reg | 20 min | 8.7/4.1 |
| c-Jun | Transcriptional | 20 min | 4.4/1.8 |
| N10 | Transcriptional | 1 hour | 53.9/18.7 |
| TIS21 | Cytoplasmic reg | 1 hour | 36.5/19.7 |
| LRG21 | Transcriptional | 1 hour | 35.4/12.5 |
| PC4 | Cytoplasmic reg | 1 hour | 10.5/2.8 |
| Sim PC4 | Cytoplasmic reg | 1 hour | 10.4/3.2 |
| KC | Secreted | 1 hour | 10.1/1.0 |
| MARC | Secreted | 1 hour | 9.3/8.8 |
| IkB | Cytoplasmic reg | 1 hour | 7.2/1.0 |
| ERG□ | Transcriptional | 1 hour | 7.1/3.9 |
| RhoB | Cytoplasmic reg | 1 hour | 6.8/2.4 |
| NAB2 | Nuclear | 1 hour | 6.3/3.8 |
| Chop 10 | Transcriptional | 1 hour | 6.2/1.0 |
| MyD118 | Nuclear | 1 hour | 6.1/3.2 |
| eRF1 | Cytoplasmic met | 1 hour | 5.8/3.3 |
| TIS11 | Transcriptional | 1 hour | 5.7/3.0 |
| Sim. DNAJ | Cytoplasmic met | 1 hour | 4.5/3.7 |
| C/EBP □ | Transcriptional | 1 hour | 4.3/4.5 |
| Stra13 | Transcriptional | 1 hour | 4.3/2.5 |
| Idb3 | Transcriptional | 1 hour | 4.0/4.2 |
| Sim. □-Actinin | Cytoplasmic reg | 1 hour | 3.8/1.2 |
| Cish | Cytoplasmic reg | 1 hour | 3.6/6.4 |
| Tissue Factor | Transmembrane | 1 hour | 3.2/1.5 |
| IRF1 | Transcriptional | 1 hour | 3.2/0.9 |
| NF-kB p65 | Transcriptional | 1 hour | 3.0/2.2 |
| Cox 2 | Cytoplasmic reg | 2 hour | 109.4/49.7 |
| PAI1 | Secreted | 2 hour | 33.4/14.8 |
| Thbs1 | Matrix | 2 hour | 32.8/21.4 |
| HB-EGF | Secreted | 2 hour | 26.4/19.5 |
| VEGF | Secreted | 2 hour | 24.7/14.0 |
| Epiregulin | Secreted | 2 hour | 22.8/13.4 |
| Gly96 | Transmembrane | 2 hour | 13.0/7.8 |
| CD44 | Transmembrane | 2 hour | 12.4/7.0 |
| IL-11 | Secreted | 2 hour | 12.1/6.2 |
| □5-Integrin | Transmembrane | 2 hour | 11.4/5.9 |
| Sim. TSG6 | Secreted | 2 hour | 10.0/4.4 |
| Tx01 | Unknown | 2 hour | 9.4/5.2 |
| TDAG51 | Cytoplamic reg | 2 hour | 8.7/5.5 |
| MAPkk3b | Cytoplasmic reg | 2 hour | 8.7/5.1 |
| Tenascin | Matrix | 2 hour | 8.0/6.0 |
| Sim. □5-Integrin | Transmembrane | 2 hour | 7.4/4.1 |
| MAPKAPK2 | Cytoplasmic reg | 2 hour | 6.5/3.5 |
| TIS11D | Transcriptional | 2 hour | 5.9/5.8 |
| Sim. CHX1 | Secreted | 2 hour | 5.7/3.6 |
| CTGF | Secreted | 2 hour | 4.9/2.1 |
| MyD116 | Cytoplasmic reg | 2 hour | 4.9/1.4 |
| c-Myc | Transcriptional | 2 hour | 4.8/2.5 |
| Sim. NF-kBp100 | Transcriptional | 2 hour | 4.2/3.2 |
| BcI3 | Nuclear | 2 hour | 4.2/7.6 |
| ARF2 | Cytoplasmic met | 2 hour | 4.0/3.5 |
| GT1 | Transmembrane | 2 hour | 4.0/3.4 |
| Fra1 | Transcriptional | 2 hour | 3.8/2.6 |
| MyD88 | Cytoplasmic reg | 2 hour | 3.8/2.5 |
| Mdm2 | Cytoplasmic reg | 2 hour | 3.6/2.1 |
| Sign. PSF | Nuclear | 2 hour | 3.3/2.7 |
| SNK | Cytoplasmic reg | 2 hour | 3.1/1.5 |
| JE | Secreted | 4 hour | 4.2/3.3 |
| TIMP1 | Secreted | 4 hour | 3.9/2.4 |
| LIX | Secreted | 4 hour | 3.4/1.4 |

Clustering of Gene Expression Profiles

Agglomerative algorithms such as hierarchical clustering start with each object (gene) being in a separate class. At each step, the algorithm finds the pair of the “most similar” objects, which are then merged in one new class and the process is repeated until all objects are grouped. Agglomerative algorithms produce a very large number of clusters when several thousands objects are involved in the data set.

One common problem with the interpretation of clustered data is to determine the “true” number of clusters. Agglomerative algorithms do not offer explicit “stopping rules” for determining the globally optimal number of classes but rather present the entire set of clusters to the user, who then has to decide on the proper degree of structure in the data.

In this example, we have used a partitioning k-means clustering algorithm to cluster the gene expression profiles iteratively into a maximum of 50 classes. This algorithm can produce a globally optimal solution since it starts with the entire data set. At each step of the algorithm the least homogeneous cluster is sub-partitioned and the process is repeated until a criterion for cluster “compactness” is met. Cluster homogeneity, or compactness, is based on the concept of fitness. The later is defined as the sum of distances observations from their corresponding cluster centroid, or

$$Fitness ( C ) = {\sum }_{ k = 1}^{C} {\sum }_{i = 1}^{{N}_{k}} d ( {X}_{ik} , \overline{{X}_{k}} ) \qquad ( 1 )$$

where Xᵢₖ is the I-th observation vector assigned to the k-th cluster, Xₖ is the vector of the k-th cluster centroid, Nₖ is the number of observations, or size, of the k-th cluster, C is the number of clusters, and d(x,y) is the distance metric (typically the Euclidian distance) between two vectors. The fitness is largest for C=1 (entire population) and monotonically approaches zero as C approaches N, the total number of observations.

Cluster homogeneity is defined now as:

$$H ( c ) = [ 1 - \frac{Fitness ( c )}{Fitness ( 1 )} ] \times 100 \qquad ( 2 )$$

that takes asymptotically the value of 100%. The optimal number of clusters C*<N is found at a homogeneity level of less than 100, depending on the internal structure of the data.

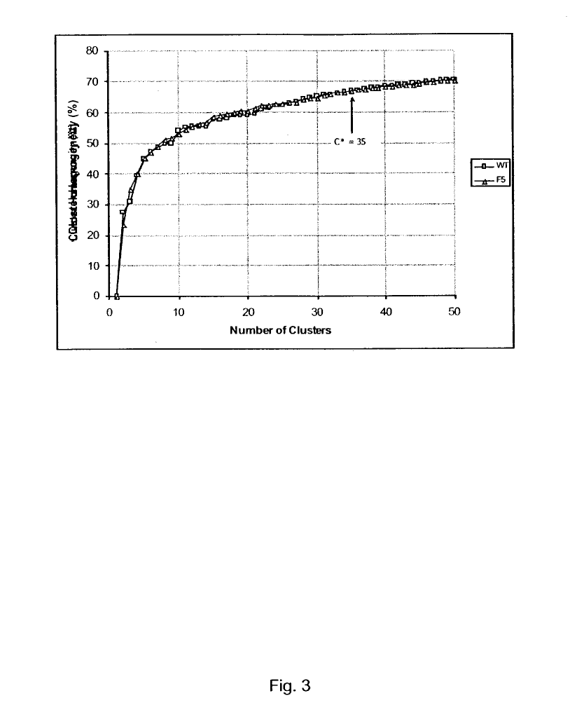

The cluster homogeneity results from clustering of the gene expression data for the wild-type and mutant strains are shown in FIG. 3. For the given settings, the algorithm arrives at an optimal number of 35 clusters.

Wild-Type Strain

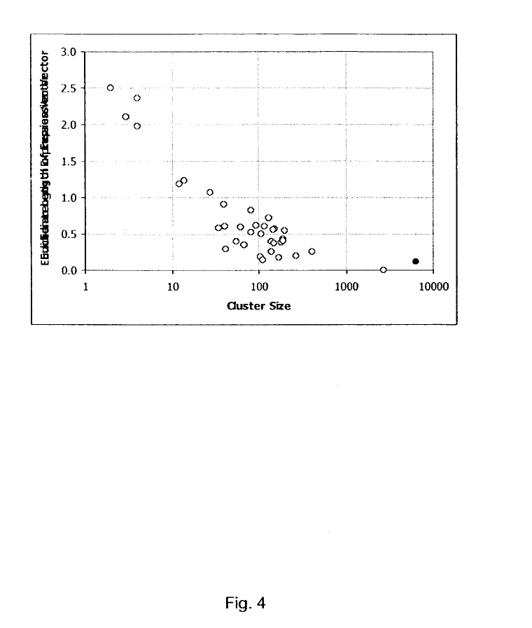

Genes are grouped in 35 clusters, which ranged in size between 2 and 2719 genes per cluster. A measure of the average expression level of the genes in each cluster, as expressed by the Euclidean length of the cluster centroid, is shown as a function of cluster size in FIG. 4.

As can be seen from the plot, a very large cluster consisting of 2179 genes (43.1% of total) exhibited expression levels almost identical to the control (length=0). On the other end, only 4 small clusters each containing at most 4 genes exhibited high expression levels throughout the time course (length>2). Finally, most of the gene clusters have moderate expression levels (length<1) and fall in the middle of the chart with size ranging between 50 and 200 genes per cluster.

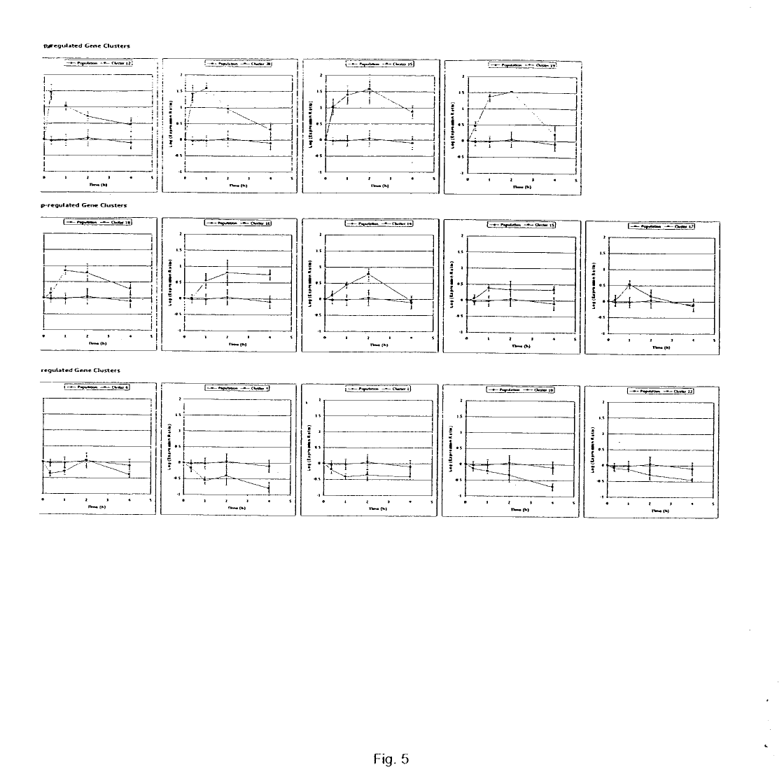

Clusters are further sub-divided into the following categories based on their expression patterns: (1) early up-regulated (higher induction than population mean at 20 minutes); (2) late up regulated (higher induction than population mean from 1 hour onwards); (3) down-regulated (lower induction than population mean); and (4) others. The typical expression “signatures” for clusters in the above three categories are shown in FIG. 5.

Early up-regulated genes exhibit a high level of expression at 20 min, indicating that these genes are IEGs, i.e. their induction does not require protein synthesis but involves latent transcriptional activators already present in the cell. Transcription of genes falling in the second category of late up-regulated genes most likely requires protein synthesis, as the expression level of these genes peaks after 1 hr from the stimulation event. Equally important are the genes falling in the last category whose expression was repressed as a result of stimulation by the extracellular signal.

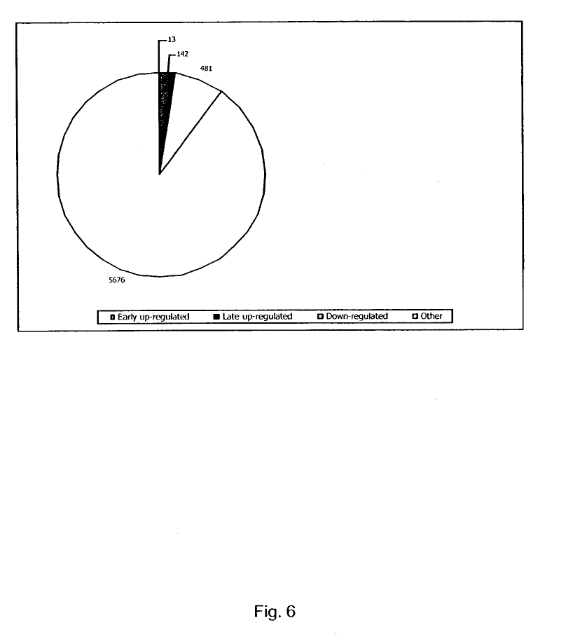

FIG. 6 shows the relative size of the clusters of genes falling in the above categories. Only 13 genes (0.2%) are early up regulated, whereas a significant number of 481 genes (7.6%) are down regulated as a result of the treatment.

F5 Mutant Strain

Comparison of the expression profiles of the wild-type strain with the mutant strain F5, which carries tyrosine to phenylalanine mutations at key binding sites for critical signaling molecules, provides some important insight regarding the degree of overlap and interaction of the various regulatory pathways.

TABLE 1.2
Comparison of Expression Patterns of the Wild Type (WT) and
Mutant (F5) Strains

| | | F5 | |
|---|---|---|---|
| Gene Clusters | WT | (genes) | WT/F5 |
| Early up-regulated | 13 | 11 | 9 |
| Late up-regulated | 142 | 144 | 44 |
| Down regulated | 481 | 578 | 215 |
| No response | 2831 | 2394 | |
| Other | 2845 | 3185 | |

The expression data from the mutant strain are analyzed in the same way. The expression patterns are similar to those of the wild type strain resulting in 34 clusters. The cluster sub-groupings for the two strains are compared in Table 1.2.

Interestingly, a similar number of genes are induced for both strains in response to the stimulant, but a larger number of genes in repressed in the mutant strain. Furthermore, it appears that the expression pattern of a larger number of genes is affected in the mutant strain compared to the wild type. This could indicate the activation of alternate or reserve pathways to compensate for the disruptions caused by the mutations.

Table 1.3 summarizes the expression profiles and functional annotations of the identified early up-regulated genes for each strain. As expected, most genes in this group code proteins that are either transcription factors or cytoplasmic regulatory proteins.

TABLE 1.3
Early Up-Regulated Genes for WT and F5 Strains

| | | | | | | | | | Gene | |
|---|---|---|---|---|---|---|---|---|---|---|
| Strain | Gene ID | 0 h | 20 min | 1 h | 2 h | 4 h | Cluster | Protein | Classification | Gene Descriptor |
| WT/F5 | M59821 | 0 | 1.559907 | 1.10551 | 0.732394 | 0.724276 | 12 | Pp92 | cytoplasmic | Mus musculus growth factor |
| | | | | | | | | | regulatory | inducible immediate early protein |
| | | | | | | | | | | (pp92) gene |
| WT/F5 | V00727 | 0 | 1.732394 | 1.174641 | 0.4843 | 0.230449 | 12 | c-Fos | transcription factor | Provirus of a replication defective |
| | | | | | | | | | | mmune sarcoma virus |
| | | | | | | | | | | (FBJ-MuSV) with c-fos(p55) and |
| | | | | | | | | | | p15 E reading frames |
| WT/F5 | X06746 | 0 | 1.093147 | 1.015064 | 0.7983935 | 0.260913 | 12 | Krox-20 | transcription factor | Mouse mRNA for Krox-20 protein |
| | | | | | | | | | | containing zinc fingers |
| WT/F5 | J03236 | 0 | 1.377283 | 1.345595 | 0.79194 | 0.013788 | 20 | JunB | transcription factor | Mus musculus transcription factor |
| | | | | | | | | | | junB (junB) gene, 5′ region and |
| | | | | | | | | | | complete cds |
| WT/F5 | M22326 | 0 | 1.857634 | 1.723456 | 1.029384 | 0.618048 | 20 | Zrf/268 | transcripiton factor | Mouse growth factor-induced |
| | | | | | | | | | | protein (zrf/268) mRNA |
| WT/F5 | M64292 | 0 | 1.284431 | 1.562887 | 0.919078 | 0.423246 | 20 | TIS21 | cytoplasmic | Mouse gluccotoid-regulated in- |
| | | | | | | | | | regulatory | flammatory prostaglandin |
| | | | | | | | | | | G/H synthase (gnPGHS) mRNA |
| WT/F5 | X16995 | 0 | 1.158362 | 1.731186 | 1.08636 | 0.230449 | 20 | N10 | transcription factor | Mouse N10 gene for a nuclear |
| | | | | | | | | | | hormonal binding receptor |
| WT/F5 | M88242 | 0 | 1.272306 | 1.675045 | 2.038859 | 1.070407 | 35 | Cox-2 | cytoplasmic | Mouse gluccatoid-regulated in- |
| | | | | | | | | | regulatory | flammatory prostaglandin |
| | | | | | | | | | | G/H synthase (gnPGHS) mRNA |
| WT/F5 | X67644 | 0 | 0.780874 | 1.115202 | 1.11583 | 0.66133 | 35 | Gly96 | transmembrane | Mmusculus gly 96 mRNA |
| WT | MB2490 | 0 | 1.470491 | 0.8119 | 0.861697 | 0.598757 | 12 | | | Mouse growth factor inducible |
| | | | | | | | | | | immediate early gene cyr 61 |
| WT | MB3960 | 0 | 0.257125 | 1.369644 | 1.523546 | 0.539269 | 19 | PAI-1 | secreted | Mouse plasminogen activator |
| | | | | | | | | | | inhibitor (PAI-1) mRNA |
| WT | MB7276 | 0 | 0.568202 | 1.188928 | 1.515874 | −0.187087 | 19 | Thbs1 | matrix | Mouse thrombospondrin (THBS1) |
| | | | | | | | | | | gene, promoter region |
| WT | U19118 | 0 | 0.544058 | 1.549616 | 1.522444 | 0.230449 | 19 | LRG-21 | transcription factor | Mus musculus transcription factor |
| | | | | | | | | | | LRG-21 mRNA |
| F5 | U39192 | 0 | 0.09691 | 0.900367 | 1.422426 | 0.969041 | 34 | HB- | secreted | Mus musculus (clone lambda |
| | | | | | | | | EGF | | mouse 1) heparin-binding EGF-like |
| | | | | | | | | | | growth factor precusor mRNA |
| F5 | X70058 | 0 | 0.274701 | 0.969733 | 0.661027 | 0.904507 | 34 | MARC | secreted | Mouse cytokine (fc) mRNA |

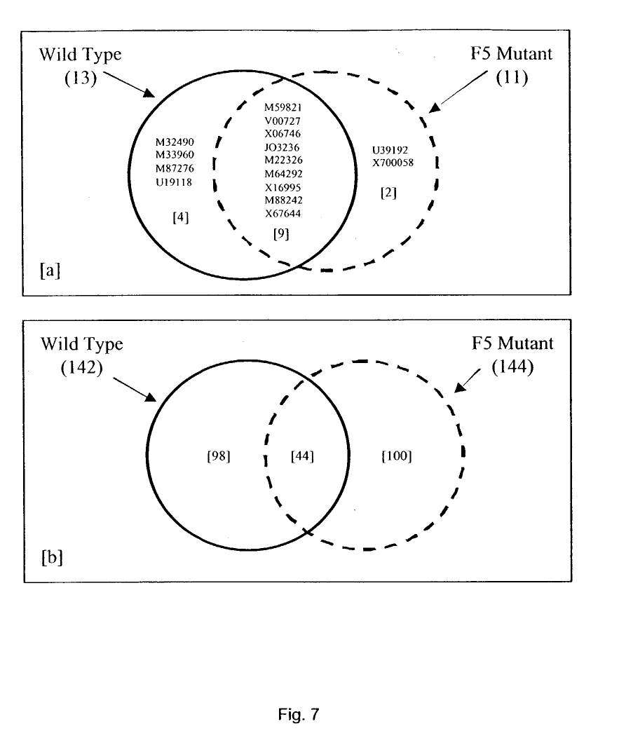

Comparison of the early-induced genes between the two strains is shown pictorially in FIG. 7(a). Nine of the 13 IEGs (69%) were common between the two strains. In all, we observed differential expression patterns in 6 IEGs: 4 IEGs from the WT strain were not induced in F5, whereas a new set of two IEGs was observed in the mutant strain. This indicates that alternate signaling pathways might be active to transduce signals and activate the early response genes. However, these pathways seem to highly overlap.

Although the early transcriptional response of the two strains is very similar, the late up-regulated genes show a considerably lesser degree of overlap (see FIG. 7(b)). The total number of genes following a late up-regulated induction profile is remarkably similar between the two strains, but only 44 (18%) were common genes, showing a great diversity in response pathways. Also, there were 215 (26%) common genes among the down-regulated clusters.

Finally, correlation analysis of the early up-regulated genes for the two strains is carried out to evaluate similarities in the expression profiles of the entire 15 genes. As shown in FIG. 6, there is a strong correlation between the same genes in the two strains (diagonal of the array), even for those genes that are classified as belonging to the IEGs for only one of the two strains (compare with FIG. 7(a)). Furthermore, the non-common IEGs can be discerned based on differences in their expression patterns relative to the other genes. These are concentrated towards the lower correlation quadrants of the array (top right corner).

The tools of clustering and correlation analysis are shown to be valuable in identifying and characterizing subtle differences in the expression profiles of biological systems. These techniques would potentially impact comparative genomics studies, especially when proteomic data are available for further elucidation of physiological pathways.

Signaling Pathways within Clusters of Early Up-regulated Genes

Using the prior art, it is demonstrated that current programs for signaling network analysis lack the functional dimension of the present invention. This deficiency limits the success of any pathway-finding program when using newly developed data rather than data from known pathways. The pathway finding operation described at http//geo.nihs.go.jp/csndb/batch_search.html, is used within the gene clusters for early up-regulated genes listed in Table 1.4. Although the database contains only human pathways, the proteins identified by the gene cluster analysis are all listed in the database, indicating human analogs.

TABLE 1.4
Gene Clusters for Early Up-Regulated Genes in WT 3T3 Cells at 20 Minutes

| GeneBank | | | Protein | |
|---|---|---|---|---|
| ID | Cluster | Protein | Classification | Gene Descriptor |
| M32490 | 12 | Cyr61 | secreted | Mouse growth factor inducible immediate early |
| | | | | gene cyr61 |
| M59821 | 12 | Pip92 | cytoplasmic regulatory | Mus musculus growth factor inducible |
| | | | | immediate early protein (pip92) gene |
| V00727 | 12 | c-Fos | transcription factor | Provirus of a replication defective munne sarcoma |
| | | | | virus (FBJ-MuSV) with c-fos(p55) and p15 E |
| | | | | reading frames |
| X06746 | 12 | Krox-20 | transcription factor | Mouse mRNA for Krox-20 protein containing |
| | | | | zinc fingers |
| M33960 | 19 | PAI-1 | secreted | Mouse plasminogen activator inhibitor (PAI-1) mRNA |
| M87276 | 19 | Thbs1 | matrix | Mouse thrombospondin (THBS1) gene, |
| | | | | promoter region |
| U19118 | 19 | LRG-21 | transcription factor | Mus musculus transcription factor LRG-21 mRNA |
| J03236 | 20 | JunB | transcription factor | Mus musculus transcription factor |
| | | | | junB (junB) gene, 5′ region and complete cds |
| M22326 | 20 | Zif/268 | transcription factor | Mouse growth factor-induced protein (zif/268) mRNA |
| M64292 | 20 | TIS21 | cytoplasmic regulatory | Mouse TIS21 gene |
| X16995 | 20 | N10 | transcription factor | Mouse N10 gene for a nuclear hormonal |
| | | | | binding receptor |
| M88242 | 35 | Cox-2 | cytoplasmic regulatory | Mouse glucocortoid-regulated inflammatory |
| | | | | prostaglandin G/H synthase (gnPGHS) mRNA |
| X67644 | 35 | Gly96 | transmembrane | M musculus gly96 mRNA |

The batch search for pathways found no pathways for cluster 12, 19, 20 or 35 gene expression data. This negative result is expected for the reasons previously discussed. The lack of functional data significantly limits the breath of inference from gene expression data. As indicated in Example 2, however, the addition of even small data sets of functional data dramatically increases the information derived from gene microarray experiments.

Example 2

The present example delineates the physiological processes and signaling pathways activated through growth factor receptors. This example illustrates that gene expression and proteomic data gathered following cellular stimulation can be interpreted in mechanistic terms by comparing the gene expression profiles to post-translational modifications of proteins with algorithms for determining linkages and associations. Such linkages and associations are then useful for identifying critical cellular pathways employed in complex cellular response mechanisms.

Methods

General methods for cell culture, stimulation, and preparation of RNA are performed as described in Example 1. Additional equipment for proteomic analysis is described.

Equipment for SDS-PAGE includes a Mini Vertical Gel System (Savant Model #MV120, Holbrook, N.Y.) and power supply (Savant Instruments Model #PS250, Holbrook, N.Y.). Supplies and reagents for western blotting are 10-20% precast gradient mini-gels (BioWhittaker Molecular Applications Catalog #58506, Rockland, Me.), 2×sample buffer (Sigma Catalog #L-2284, St. Louis, Mo.), beaker, 1000 mL (VWR Catalog #13910-289, Rochester, N.Y.), color molecular weight standard (Sigma Catalog #C-3437, St. Louis, Mo.), glycine (Sigma Catalog #G-7403, St. Louis, Mo.), graduated cylinder, 1000 mL (VWR Catalog #24711-364, Rochester, N.Y.), microfuge tubes, 0.5 mL Safe-Lock (Brinkmann Catalog #22 36 365-4, Westbury, N.Y.), microfuge tubes, 1.7 mL (VWR Catalog #20172-698, Rochester, N.Y.), pipet tips for 2-20 μL and 20-200 μL pipettors (VWR Catalog #53512-260, Rochester, N.Y.), pipet tips, gel loading (VWR Catalog #53509-018, Rochester, N.Y.), sodium dodecyl sulfate (SDS) (Sigma Catalog #L-4509, St. Louis, Mo.), Stir Bar, Magnetic (VWR Catalog #58948-193, Rochester, N.Y.), storage bottle, 1000 mL (Corning Catalog #1395-1L, Corning, N.Y.), and trizina Base (Sigma Catalog #T-6066, St. Louis, Mo.).

Prepare 5×SDS-PAGE buffer by dissolving 15 grams of Tris base, 72 grams glycine, and 5 grams SDS in 900 mL distilled water in a 1000 mL beaker with a magnetic stir bar. Place on a magnetic stirrer and stir until dissolved. Adjust volume to 1000 mL with a 1000 mL cylinder. Store at 4° C. Prepare 1×SDS-PAGE buffer by combining 200 mL of the 5×stock with 800 mL water. Store in a 1000 mL storage bottle at 4° C. Warm to room temperature before use. Melt the 2×Sample Buffer at room temperature and store as 500 μL aliquots in 1.7 mL microfuge tubes in −30° C. freezer. Assemble vertical gel system according to manufacturers guidelines. Pour enough 1×SDS-PAGE buffer into gel system to cover top of gel and enough in bottom of the apparatus to cover bottom of glass plates. Remove a tube of 2×Sample Buffer from freezer and melt at room temperature. Melt frozen cell lysate samples on ice. Dilute cell lysate samples 1:1 with 2×sample buffer in 0.5 mL Safe-Lock tubes (15 μL of cell lysate sample and 15 μL 2×Buffer). Put remaining 2×Sample Buffer back into freezer (−30° C.). Put cell lysate samples back into freezer (−80° C.). Heat protein samples and molecular weight standards (if required) to 95-100° C. for 5 minutes. Briefly, spin in microfuge to collect sample at bottom of tube, and load equal amounts of protein in wells of pre-cast gel. Run at 30 mA per gel at constant current for 60 minutes or until dye reaches the bottom of gel.

Supplies and reagents for western blotting of phosphotyrosyl proteins includes anti-phosphotyrosine antibody 4G10 (UBI Catalog #05-321, Lake Placid, N.Y.), Blotting Paper (VWR Catalog #28303-104, Rochester, N.Y.), glycine (Sigma Catalog #G-7403, St. Louis, Mo.), hydrochloric acid (HCl) (VWR Catalog #VW3110-3, Rochester, N.Y.), methanol (VWR Catalog #VW4300-3, Rochester, N.Y.), NaOH (Sigma Catalog #S-5881, St. Louis, Mo.), nitrocellulose membrane (Schleicher & Schuell Catalog #10402680, Keene, N.H.), Nonfat dry milk (Carnation Brand), peroxidase labeled goat anti-mouse IgG (KPL Catalog #474-1806, Gaithersburg, Md.), and phosphate buffered saline (PBS) (Mediatech Catalog #21-040-CV, Herndon, Va.).

Perform SDS-polyacrylamide gel electrophoresis for phosphotyrosine proteins on cell lysate sample as in Example 1. Remove membrane from glass plates and equilibrate in Towbin buffer for 5 minutes with gentle rotation at room temperature. Cut nitrocellulose membrane to correct size, nicking off the lower right hand corner. Prewet membrane with ultra-pure water, then equilibrate for 5 minutes in transfer buffer. Prewet 6 pieces of blotting paper for each gel to be transferred in 1×Towbin buffer.

Set up transfer sandwich according to the manufacturer's directions. Transfer proteins at 96 mA per gel for 60 minutes per gel. Check for good protein transfer by staining with 10 mL Ponceau S solution for 5 minutes, then washing several times with water. Block the blotted membrane with 10 mL of freshly prepared PBS containing 3% nonfat dry milk (PBS-NFDM) for 20 minutes at room temperature with constant agitation. Incubate the membrane with the primary antibody diluted to 1 μg/mL in 5 mL freshly prepared PBS-NFDM overnight at 4oC and sealed in a plastic bag.

Wash the membrane twice with water. Incubate the membrane in the secondary antibody diluted 1:3000 in 10 mL freshly prepared PBS-NFDM for 1.5 hours at room temperature with constant agitation. Wash the membrane twice with water. Wash the membrane in PBS-0.05% Tween 20 for 3.5 minutes at room temperature with constant agitation. Wash membrane 3-4 times with water. Detect tyrosine phosphoproteins using chemiluminescence.

Chemiluminescence for visualization of phosphotyrosine proteins is performed using a UVP darkroom with cooled integrated camera (Epi Chemi II Darkroom with LabWorks Software, UVP, Upland, Calif.), LumiGlo® Chemiluminescent Substrates A and B (KPL Catalog #54-61-02, Gaithersburg, Md.). Remove LumiGlo® Chemiluminescent Substrates A and B from refrigerator. After proteins have been blotted to nitrocellulose or PVDV, drain excess water from membrane by touching edge of membrane on a clean KimWipe. Place membrane into a clean weigh boat or other suitable container. Add 0.8 mL of Substrate A and of Substrate B directly to membrane and swirl around to mix. Put LumiGlo® Chemiluminescent Substrates A and B back into refrigerator. Allow substrate to incubate on membrane for 1 minute at room temperature. Remove membrane from weigh boat, drain off excess substrates, and place directly onto the transilluminator of the Epi Chemi II system. In the LabWorks program provided, select On-Chip Integration and integrate for various times until a good signal is achieved (1,3,6,10 and/or 15 minutes, depending on how much protein of interest is present on membrane). Using the software, identify bands of interest and print out the Integrated Optical Density of these bands.

Data Analysis:

1. For each protein band intensity measurements are first normalized to magnitude of 1 across the time profile. Data can also be normalized across protein bands to a magnitude of 1 at each time point. p1 2. Partitioning k-means clustering is applied to the normalized data as explained in Example 1. Optimal number of clusters was determined to be 5.

3. Average profiles are calculated for the proteins within each cluster.

4. Protein clusters are grouped according to the dynamics accumulation to early or late phosphorylated clusters.

The similarity of the proteomic clusters to the genomic expression clusters is then determined through association analysis based on a similarity measure, as for example the Pearson's correlation coefficient or Euclidean distance of the two profiles.

TABLE 2.5
Quantification of Protein Tyrosine Phosphorylation in
M-CSF-Treated 3T3 Cells

| | | |
|---|---|---|
| Distance | Molecular Wt | Intensity |

| Band | [cm] | [kDa] | 0 h | 20 m | 1 h | 2 h | 4 h |
|---|---|---|---|---|---|---|---|
| 1 | 0.8 | 134.9 | 0 | 0 | 389.9 | 383.4 | 381.9 |
| 2 | 1.4 | 109.5 | 0 | 0 | 223.1 | 211.4 | 221.3 |
| 3 | 2 | 93.3 | 0 | 32.2 | 0 | 0 | 0 |
| 4 | 2.35 | 86 | 0 | 0 | 196.8 | 183.4 | 190.8 |
| 5 | 2.9 | 76.4 | 0 | 55 | 0 | 0 | 0 |
| 6 | 3.55 | 67.2 | 0 | 0 | 0 | 0 | 174 |
| 7 | 4.4 | 57.5 | 0 | 0 | 80.9 | 85.8 | 182.3 |
| 8 | 5.1 | 50.8 | 0 | 61.3 | 0 | 0 | 0 |
| 9 | 5.38 | 48.4 | 0 | 0 | 151.3 | 159.7 | 157.2 |
| 10 | 5.9 | 44.2 | 0 | 0 | 271.1 | 254.5 | 253.1 |
| 11 | 6.7 | 38.4 | 0 | 0 | 91.4 | 116.5 | 219.9 |
| 12 | 7.3 | 34.5 | 0 | 0 | 0 | 47.7 | 75.4 |
| 13 | 7.5 | 33.3 | 0 | 0 | 75.6 | 54.4 | 73.2 |
| 14 | 7.51 | 33.2 | 0 | 0 | 0 | 49.3 | 0 |
| 15 | 8.4 | 28.2 | 0 | 0 | 39.7 | 95.5 | 177.4 |
| 16 | 8.85 | 25.8 | 0 | 0 | 0 | 0 | 68.9 |
| 17 | 9.5 | 22.6 | 0 | 0 | 51.4 | 0 | 168.4 |
| 18 | 10 | 20.2 | 0 | 0 | 52.1 | 48.8 | 42.5 |
| 19 | 10.15 | 19.6 | 0 | 36.3 | 0 | 0 | 0 |
| 20 | 10.4 | 18.5 | 0 | 0 | 123.7 | 119.6 | 112.4 |
| 21 | 10.9 | 16.3 | 0 | 32.6 | 114 | 110.5 | 123.8 |

Clustering of Proteomic Profiles

The k-means algorithm determined an optimal number of 5 clusters. The distribution of the proteomic clusters is shown in FIG. 2.1.

Cluster A is the largest cluster containing 11 of the 21 visible phosphorylated protein bands. Cluster B is the smallest containing only 1 protein band, which has a unique profile compared to the other bands (see FIG. 2.2).

The results of the clustering algorithm indicated that the phosphorylation profiles of all proteins were the most dissimilar at 1 and 2 h, and the most similar at 4 h. This clearly has implications on experimental design in this system, suggesting that if a single time-point design is to be followed, the proteomic measurement should be taken at 1 or 2 h after stimulation.

The time profiles of the phosphorylated protein clusters are shown in FIG. 2.2. The total amount of phosphorylated protein (sum of intensity of all bands) is also shown for comparison. As can be seen, clusters E and C contain proteins that are phosphorylated as early as 20 min after addition of the stimulant. In particular, cluster E contains three proteins with molecular weights 93.3, 76.4 and 50.8 kDa that seem to have a role in the early stages of the signal transduction process.

Association Analysis of Gene and Proteomic Profiles

Separate analyses of gene expression and proteomic data resulted in classification of the different genes and phosphorylated proteins according to the dynamic profiles of their levels after stimulation with M-CSF. The gene expression clusters in particular identified groups of genes that showed high levels of induction, prior to protein synthesis. Similarly, two of the protein clusters showed early phosphorylation, suggesting that these proteins might be related somehow to the immediate early induced genes. If this analysis is extended to the entire set of gene expression and proteomic clusters, the association between protein phosphorylation and gene expression can be mapped out.

In the following analysis, the similarity of the gene expression and proteomic profiles was evaluated based on Pearson's correlation coefficient, which is defined as:

$${ρ}_{XY} = \frac{1}{N} {\sum }_{i = 1}^{N} ( \frac{{X}_{i} - \overline{X}}{{s}_{X}} ) ( \frac{{Y}_{i} - \overline{Y}}{{s}_{Y}} ) \qquad ( 3 )$$

where X is the expression profile of a gene cluster, Y is the expression profile of a protein cluster, N is the number of time points, and \overline{X} and sₓ are the average and standard deviation of the values in each profile.

The results of this analysis are shown in FIG. 2.3. The figure shows the color-coded map of associations. The actual values of the correlation coefficient are also shown. To make the visual inspection clearer, the resulted correlation matrix was clustered in both directions and the rows and columns were re-arranged according to the results of the clustering.

From the visual inspection of the proteomic-genomic association matrix several areas of positive (red) or negative (green) association between the clusters is evident. For example, gene clusters 12, 20, and 35, which are early-regulated clusters, show a negative association with protein cluster A, indicating opposite regulation. Also, gene cluster 9 (containing 56 genes) shows a strong positive association with protein clusters C and E.

Further analysis of cluster 9 gene products with cluster E proteins using our protein database indicates an association of M-CSF with early response proteins PTP-1C and Shc. Both of these proteins are cytoplasmic tyrosine phosphatases. In our protein dataset, a network signaling linkage from PTP-1C is identified with the tyrosine phosphorylation of a 65-kDa cytoplasmic protein pp65.

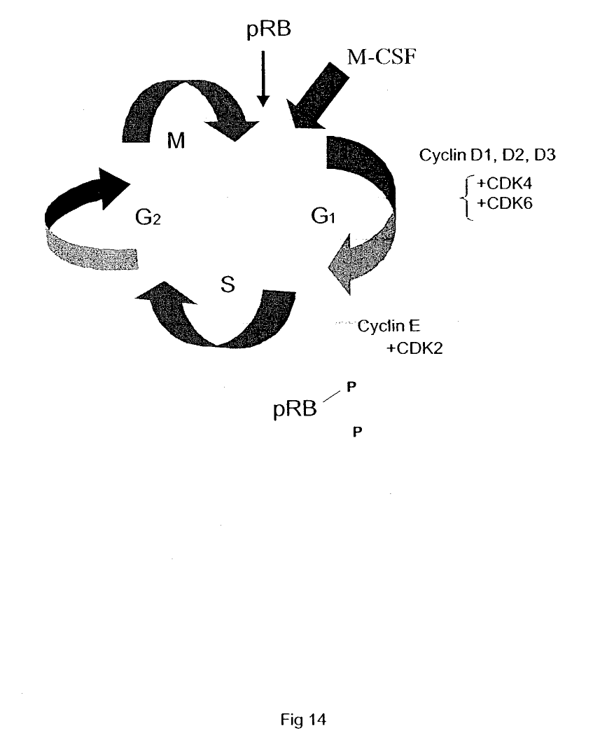

Estimating signaling associations among signaling pathways within the gene cluster 9 and protein cluster E overlap, it is discovered that the highest degree of association (0.125) is achieved with cell cycle regulatory proteins (see FIG. 14). These include cyclins D1, D2, D3 and E, cyclin dependent kinases CDK4/6/2 and RB protein. While further analyses of time sequences is not presented, an interesting strong down-regulation of the p53 protein is identified by the present invention at 1 hour followed by a stronger up-regulation by 4 hours.

As a knowledge-based system, information of associations in one series of experiments may be combined with other experiments to continue to improve the strength of association of adjacent molecules and pathways. Other post-translational processes added to the experimental design will also function to improve the strength of pathway identifications. This example illustrates that the combination of gene expression data and structure/function protein assessment with a structure/function protein database described by the present invention generates superior information relating to signaling networks and is more useful to the discovery of novel pathways.

We claim:

1. A method of identifying a relationship between gene expression and protein modifications in a cell comprising
a. determining gene expression generated in the cell,
b. determining protein modifications generated in the cell, and
c. coordinating the gene expression and protein modifications generated in the cell,
thereby identifying the relationship between gene expression and protein modifications.

2. The method of claim 1, wherein gene expression is determined using a cDNA microarray.

3. The method of claim 1, wherein gene expression is determined by Serial Analysis of Gene Expression (SAGE).

4. The method of claim 1, wherein gene expression is determined using northern blot analysis of gene transcription.

5. The method of claim 1, wherein gene expression is determined by analysis of chemically modified nucleic acids.

6. The method of claim 1, wherein determination of protein modifications comprises a determination of alterations in protein expression.

7. The method of claim 1, wherein determination of protein modifications comprises a determination of post-translational modifications.

8. The method of claim 7, wherein the post-translational modification is phosphorylation, glycosylation, or methylation.

9. The method of claim 1, wherein the protein modifications are determined using one-dimensional gel electrophoresis.

10. The method of claim 9, wherein the one-dimensional gel electrophoresis is accomplished with or without affinity purification.

11. The method of claim 1, wherein the protein modifications are determined using microchips coated with antibodies.

12. The method of claim 1, wherein the protein modifications are determined using identification of non-denatured protein/protein complexes in solution.

13. The method of claim 1, wherein the post-translational modifications are determined using functional assays for enzyme activity.

14. The method of claim 1, wherein the protein modifications are determined using bioassays for cytokines or receptor/ligand binding.

15. The method of claim 1, wherein the protein modifications are determined through localization of proteins within the cell.

16. The method of claim 1, wherein the protein modifications are determined through large-scale mouse knockouts.

17. The method of claim 1, wherein the protein modifications are determined through large-scale animal assays for functional proteins.

18. The method of claim 1, wherein the protein modifications are determined through differential display by two-dimensional gels.

19. A method of investigating a metabolic pathway comprising
a. exposing a cell to an agent involved in the metabolic pathway and
b. identifying the relationship between gene expression and protein modifications generated in the cell in response to the agent according to the method of claim 1,
thereby investigating the metabolic pathway.

20. A method of typing a diseased cell comprising
a. identifying the relationship between gene expression and protein modifications in the diseased cell according to the method of claim 1,
b. identifying the relationship between gene expression and protein modifications in a corresponding normal cell according to the method of claim 1, and
c. comparing the coordinated gene expression and protein modifications of the diseased cell with the normal cell,
thereby typing the diseased cell.

21. A method of identifying biological activity of one or more test materials comprising
a. exposing a cell to the one or more test materials and
b. identifying the relationship between gene expression and protein modifications generated in the cell in response to exposure to the one or more test materials according to the method of claim 1,
thereby identifying the biological activity of the one or more test materials.

22. A method of comparing a combination of different test materials comprising
a. identifying the biological activity of one or more test materials according to the method of claim 21,
b. identifying the biological activity of one or more test materials according to the method of claim 21, wherein the one or more test materials in step a is different from the one or more test materials in step b,
c. comparing the biological activity identified in step a with the biological activity identified in step b.

23. A computer system for identifying the relationship between gene expression and protein modifications comprising
a. a database including records comprising
i gene expression data and
ii. protein modifications data,
b. one or more algorithms for statistically analyzing the gene expression and protein modifications data,
c. one or more algorithms for coordinating the statistically analyzed gene expression and protein modifications data,
d. a system for output and presentation of the results from the algorithms,
e. a repository systems to index and stored the database and results, and
f. a query system for retrieval of database and results.

24. A computer-based system for predicting the relationship between gene expression and functional protein expression comprising
a. a database management system for storing gene expression data and protein modification data;
b. a database system for aggregating information about individual genes and proteins, including chromosomal location, function, pathway membership, phosphorylation status.
c. algorithms for correcting experimental data for experimental biases
d. one or more clustering algorithms for extracting patterns
i. from gene expression profiles, and
ii. from functional protein expression profiles;
e. one or more algorithms for extracting relationships between gene expression patterns and functional protein expression patterns;
f. algorithms for annotating gene expression profiles to derive functional characterization of gene expression or protein expression response
g. a repository for storage of derived relationships; and
h. a query system for retrieval of discrete patterns, relationships and experimental conditions.

25. A computer-readable storage medium comprising digitally encoded data, wherein the data comprise results of coordination of gene expression and protein modification.
